(19) United States
(12) Patent Application Publication
(10) Pub. No.: US 2023/0321303 A1
(43) Pub. Date: Oct. 12, 2023

(54) VOLATILE COMPOSITION DISPENSER WITH VISUAL INDICATOR

(71) Applicant: The Procter & Gamble Company, Cincinnati, OH (US)

(72) Inventors: Rahul VYAS, Singapore (SG); Desmond NG, Singapore (SG); Mei San Gigi CHO, Singapore (SG); William Michael CANNON, West Harrison, IN (US)

(21) Appl. No.: 18/128,268

(22) Filed: Mar. 30, 2023

Related U.S. Application Data
(60) Provisional application No. 63/329,479, filed on Apr. 11, 2022.

(51) Int. Cl. A61L 9/12; A61L 9/04; A01M 29/12

(52) U.S. Cl. CPC A61L 9/12; A61L 9/042; A01M 29/12

(57) ABSTRACT

A volatile composition dispenser including a solid article having a volatile composition and a peripheral evaporative surface. The solid article shrinks relative to the peripheral evaporative surface from an initial size to a reduced size smaller than the initial size upon exposure of the solid article to the environment in an interior space. The solid article is characterized by a shrinkage in a direction away from the peripheral evaporative surface of greater than 1% to less than 40%. A visual indicator is located proximal to the peripheral evaporative surface of the solid article. The dispenser has a first configuration in which the visual indicator is not visually perceptible, and a second configuration in which the visual indicator is visually perceptible upon shrinkage of at least a portion of the solid article from the initial size to the reduced size for indicating a state of use of the dispenser.

### FIELD OF THE INVENTION

The present invention relates to volatile composition dispenser for delivering a volatile composition, and particularly relates to air fresheners with a visual indicator for providing a visual product life signal.

### BACKGROUND OF THE INVENTION

The use of various devices for diffusion of volatile compounds such as perfumes, sanitizing agents, insect repellants, air freshening compositions, deodorizers are well known. For example, consumers buy air fresheners to freshen their homes. Conventional air fresheners release an air freshening composition into the surroundings for a period of time until the freshening composition is depleted. However, such air fresheners seldom offer adequate visual indication to the consumer that replacement is required before or after the freshening composition is depleted. Accordingly, consumers crave a visual cue that would make it easier to see when it is time for a replacement when the freshening composition in the air freshener is almost finished.

A gel air freshener device that provides a visual cue is described in International Publication No. WO03/008000A1 (Givaudan SA). WO03/008000A1 describes a gel air freshener device that makes use of the shrinkage of the gel matrix to move segments of the device thereby providing a visual cue as to when the gel matrix is exhausted. However, it is complicated to manufacture and may require assembly of the segments to be aligned properly so that the device can achieve the function of providing the visual cue.

U.S. Pat. No. 10,918,756B2 describes a solid non-aqueous gel air odorizer which has a combination of ingredients that provide consistent delivery of active fragrance, deodorant, or insect repellant active over an extended period (at least 30 days; preferably, at least 50 days) and transitions from opaque to clear as the active dissipates. However, it may not be intuitive for users to associate a clear color of the gel with no, or little remaining active in the odorizer without user operating instructions.

Notwithstanding the above, there remains a need for a volatile composition dispenser that is intuitive and user friendly to guide the user in determining when a replacement is required before or when the dispenser is depleted.

### SUMMARY OF THE INVENTION

The present invention relates to a volatile composition dispenser comprising: a solid article comprising a volatile composition and a peripheral evaporative surface, wherein the solid article is capable of shrinkage relative to the peripheral evaporative surface from an initial size to a reduced size smaller than the initial size upon exposure of the solid article to the environment in an interior space wherein the solid article is characterized by a shrinkage in a direction away from the peripheral evaporative surface of greater than 1% to less than 40%; and a visual indicator located proximal to the peripheral evaporative surface of the solid article; wherein the dispenser has a first configuration wherein the visual indicator is not visually perceptible, and a second configuration wherein the visual indicator is visually perceptible upon shrinkage of at least a portion of the solid article from the initial size to the reduced size for indicating a state of use of the dispenser.

### BRIEF DESCRIPTION OF DRAWINGS

FIG. 1 is a front view of a volatile composition dispenser with a solid article having an initial size and a visual indicator in a first configuration in which the visual indicator is not visually perceptible according to the present invention;

FIG. 2 is a front view of the volatile composition dispenser of FIG. 1 in a second configuration in which the visual indicator is visually perceptible;

FIGS. 3A, 3B, 3C are schematic drawings illustrating a method of providing a visual product life signal for a volatile composition dispenser for delivering a volatile composition in an interior space according to the present invention;

FIG. 4 is a front perspective view of a solid article with at least one textured surface for a volatile composition dispenser according to the present invention;

FIG. 5 is a front perspective view of a solid article substantially free of a textured surface for a volatile composition dispenser according to the present invention;

FIG. 6 is a front perspective view of an alternative embodiment of a volatile composition dispenser with a solid article having an initial size and a visual indicator in a first configuration in which the visual indicator is not visually perceptible according to the present invention;

FIG. 7 is a front view of the volatile composition dispenser of FIG. 6 in a second configuration in which the visual indicator is visually perceptible;

FIG. 8 is a perspective view of components in the volatile composition dispenser of FIG. 6;

FIG. 9 is a front perspective view of an alternative embodiment of a volatile composition dispenser with an article holder and a solid article according to the present invention;

FIG. 10 is a front perspective view of an alternative embodiment of an article holder for a volatile composition dispenser according to the present invention; and

FIG. 11 is a front perspective view of the article holder of FIG. 10 with a solid article according to the present invention.

### DETAILED DESCRIPTION OF THE INVENTION

It has been found that end of product life signals in consumer products play an important role in enabling consumers/users to determine when to make a new purchase or to refill the product. In particular, it is difficult for users of volatile dispensing products for delivering a benefit in an interior space to assess whether the volatile composition is exhausted. In particular, for scented volatile compositions, absence of scent may be regarded as an indicator that the perfume is exhausted. However, for non-scented volatile compositions that deliver a benefit without providing a scent (non-scented benefit) such as malodor removal, deodorizing, insect repelling, antibacterial efficacy or the like, physical depletion of the composition is often the only way to indicate end of product usage.

The present invention relates to a volatile composition dispenser with a visual product life signal, and a method of providing a visual product life signal for a volatile composition dispenser for delivering a volatile composition in an interior space. Specifically, the volatile composition dispenser comprises a solid article comprising a volatile composition and a peripheral evaporative surface. The solid article is capable of shrinkage from an initial size to a reduced size smaller than the initial size upon exposure of the solid article to the environment due to vaporization of the volatile composition into the atmosphere of the interior space. The shrinkage of the solid article in a direction away from the peripheral evaporative surface is greater than 1% to less than 40%. A visual indicator is located proximal to the peripheral evaporative surface of the solid article. The visual indicator is stationary and its appearance and/or position does not change during the product life of the dispenser. It has been surprisingly found that a visual product life indicator can be designed into a volatile composition dispenser by the dispenser having a first configuration wherein the visual indicator is not visible, and a second configuration wherein the visual indicator is visible upon shrinkage of the solid article from the initial size to the reduced size for providing a visual cue of a state of use of the dispenser.

In the following description, the dispenser described is a consumer product, such as an air freshener, for evaporating a volatile composition in spaces to deliver a variety of benefits such as freshening, malodor removal or scenting of air in spaces such as rooms in household and commercial establishments, or enclosed spaces such as a vehicle passenger compartment space. However, it is contemplated that the dispenser may be configured for use in a variety of applications to deliver a volatile composition to the atmosphere and the dispenser may include but is not limited to consumer products, such as, for example air freshening products.

Prior to describing the present invention in detail, the following terms are defined for clarity. Terms not defined should be given their ordinary meaning as understood by a skilled person in the relevant art.

“Horizontal orientation” as used herein, refers to a position of a volatile composition dispenser according to the present invention wherein a central evaporative surface is facing the environment in an upward or downward position.

“Solid article” as used herein, refers to a chemically cross-linked gel composition that is molded in the form of a three-dimensional object having a width, length and thickness along an x-axis, y-axis, and z-axis, respectively. The solid article is self-supporting and comprises at least two evaporative surfaces.

“Visual indicator” as used herein, refers to any element that indicates a stage in the product life cycle of a volatile composition dispenser, or a state of use of the volatile composition dispenser.

“Visually perceptible” as used herein, refers to the ability to see the visual indicator and notice information represented by the visual indicator.

“Vertical orientation” as used herein, refers to a position of a volatile composition dispenser according to the present invention wherein a central evaporative surface is facing the environment in a forward-facing position or in a rear facing position.

“Non-energized” as used herein, means that the volatile composition dispenser is passive and does not require to be powered by a source of external energy. In particular, the volatile composition dispenser does not need to be powered by a source of heat, gas, or electrical current, and the volatile composition is not delivered by aerosol means. Further, as used in this specification and the appended claims, the singular forms “a”, “an”, and “the” include plural references unless the content clearly dictates otherwise.

“Top notes” as used herein, refer to perfume raw materials having a high volatility.

“Bottom notes” as used herein, refer to perfume raw materials which are less volatile relative to the top notes.

“Vapor” as used herein, refers to a gaseous form of an organic or inorganic substance which coexists as a solid or liquid at ambient conditions including but not limited to temperature, humidity, and air pressure.

“Vapor impermeable substrate” as used herein, refers to a material configured to resist diffusion of vapor from the dispenser prior to its intended use.

“Vapor release rate” as used herein, refers to a measure of the passage of vapor through a substrate.

“Volatile composition” as used herein, refers to a material that is vaporizable at room temperature and atmospheric pressure without the need of an additional energy source. The volatile composition may be configured for various uses, including but not limited to, air freshening, deodorization, odor elimination, malodor counteraction, pest control, insect control, insect repelling, medicines/medicaments, disinfectants, sanitization, mood enhancement, aromatherapy aid, scented compositions, non-scented compositions, or any other use which requires a volatile composition that acts to condition, modify, or otherwise change the atmosphere or the environment. Further, it is not necessary for all of the component materials of the volatile composition to be volatile. Any suitable volatile composition in any amount or form, including a liquid, solid, gel or emulsion, may be used. Materials suitable for use herein may include non-volatile compounds, such as carrier materials (e.g., water, solvents, etc.). It should also be understood that when the volatile composition is described herein as being “delivered”, “emitted”, or “released”, this refers to the volatization of the volatile component thereof and does not require that the non-volatile components thereof be emitted.

For the purposes of illustrating the present invention in detail, the invention is described below as a non-energized volatile composition dispenser. However, the volatile composition dispenser may be configured for use with an energized device such as, for example, an electrical heating device or a fan. The solid article described hereinafter is a gel composition comprising a perfume as an example of a volatile composition. The gel composition is polyester polyol cross-linked using a cross-linking agent selected from the group consisting of: isocyanates, isothiocyanates and mixtures thereof. However, it will be appreciated that the solid article may be formed from any gel composition that can be molded into three-dimensional objects and that when set, is self-supporting and has at least a peripheral evaporative surface and a central evaporative surface.

The solid article may be configured in a variety of shapes and sizes to facilitate customization for use as volatile composition dispensers such as air fresheners in vehicles, residential interior spaces, commercial interior spaces, a household furniture interior space such as cupboards or lockers, a household appliance interior space. Preferably the household appliance may be selected from the group consisting of: refrigerators, air conditioners, washing machine, automatic dishwashing machine. The interior space may be a portable consumer product interior environment, preferably the portable consumer product may be bags, luggage, or the like.

### Volatile Composition Dispenser

The solid article of the present invention can be implemented using a volatile composition dispenser, such as an apparatus for delivering a volatile composition into an interior space. It is contemplated that the apparatus may be configured for use in a variety of applications to deliver volatile materials to the atmosphere and/or a surface as long as the volatile material is evaporated from the apparatus. For the purposes of this disclosure, but without intending to limit the scope of the invention, the apparatus is described as a non-energized apparatus.

FIG. 1 is a front view of a volatile composition dispenser 1 with a solid article 2 comprising a volatile composition according to the present invention. The solid article 2 is configured to contain a solid phase of the volatile composition and comprises a plurality of evaporative surfaces to allow the solid phase of the volatile composition to evaporate therefrom. The volatile composition may be selected from the group consisting of: a perfume, a deodorizing agent, a sanitizing agent, an insect repellant, a malodor reduction agent, and mixtures thereof. The volatile composition may be present in a level of 3 wt % to 85 wt %, from 15 wt % to 75 wt %, from 20 wt % to 60 wt %, or different combinations of the upper and lower percentages described above or combinations of any integer in the ranges listed above, by weight of the solid article.

Referring to FIG. 1, the solid article 2 comprises at least one central evaporative surface 4 and a peripheral evaporative surface 5 surrounding the at least one central evaporative surface 4. The peripheral evaporative surface 5 has at least one edge 6 that defines part of an outer periphery of the solid article 2. The volatile composition dispenser 1 comprises a housing 3 for housing the solid article 2. The housing 3 may comprise a housing opening 7 configured to enable the at least one central evaporative surface 4 and/or the peripheral evaporative surface 5 to be in fluid communication with an interior space. A visual indicator 8 is disposed within the housing 7 behind the solid article 2.

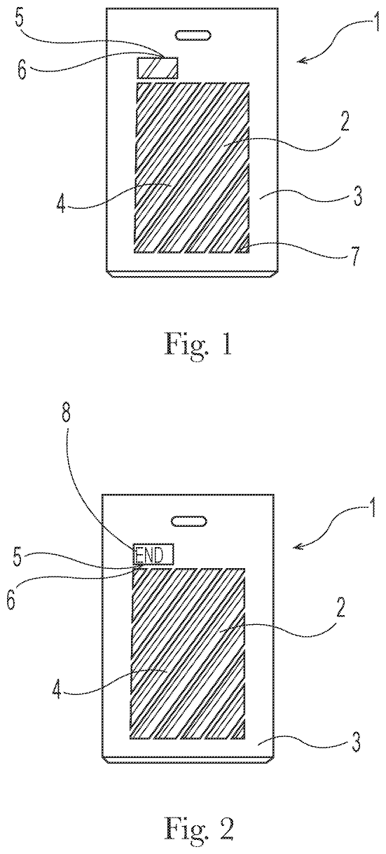

FIG. 2 is a front view of the volatile composition dispenser 1 of FIG. 1 in a second configuration in which the visual indicator 8 is visible upon shrinkage of the solid article 2 from the initial size to a reduced size for providing a visual cue of a state of use of the dispenser 1. The visual indicator 8 is located on an inner surface of the housing 3 and may be positioned anywhere in the housing as long as it is proximal to the peripheral evaporative surface 5.

The visual indicator 8 may indicate a state of the dispenser selected from the group consisting of: an end of life of the dispenser, a quantitative state indicative of a number of days use of the dispenser, and combinations thereof. As shown in FIG. 2, the visual indicator 8 may be in the form of letters defining an English word “END” which is indicative of the end of life of the dispenser. Alternatively, the visual indicator 8 may be a set of characters or a graphical symbol indicative of the state of the dispenser.

The volatile composition dispenser 1 may be configured in any orientation, such as in a vertical orientation during use such as shown in FIGS. 1 and 2.

An entire area of the central evaporative surface 4 may comprise a surface finish, such as shown in FIG. 1 and FIG. 2. A technical effect of providing a surface finish on the entire area of the central evaporative surface 4 is for ease of manufacturing, and/or hiding interior details of the housing 3. However, it will be appreciated that the visual indicator 8 may also be concealed by only texturing at least a region of the central evaporative surface 4 adjacent to the visual indicator 8, and while other regions of the central evaporative surface 4 are textured which is described hereinafter.

### Method

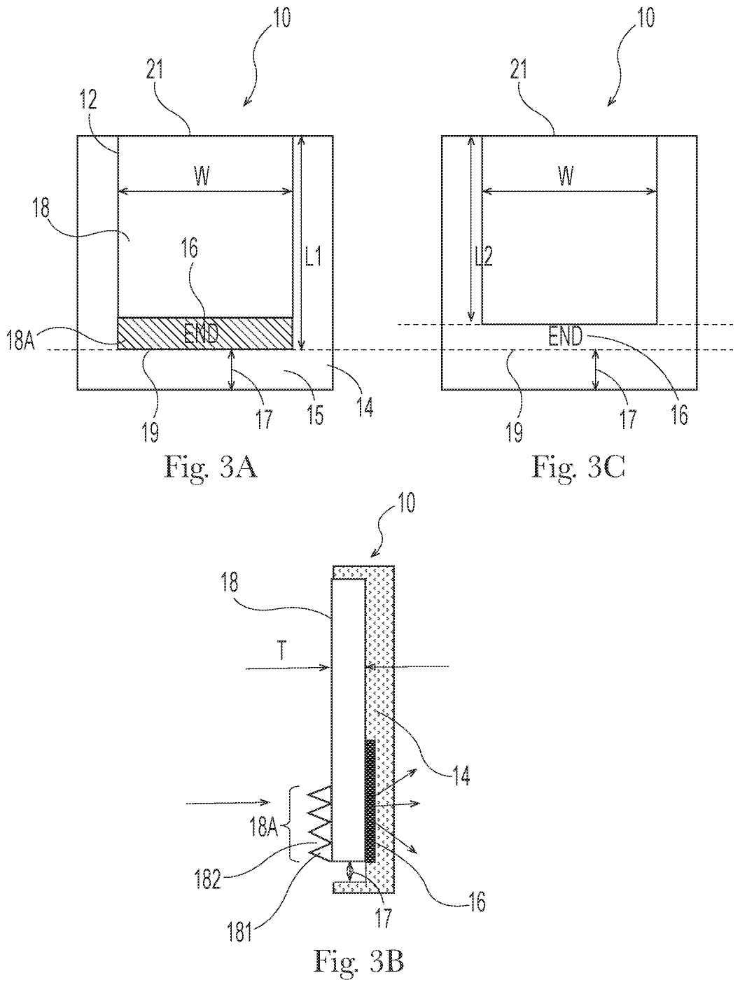

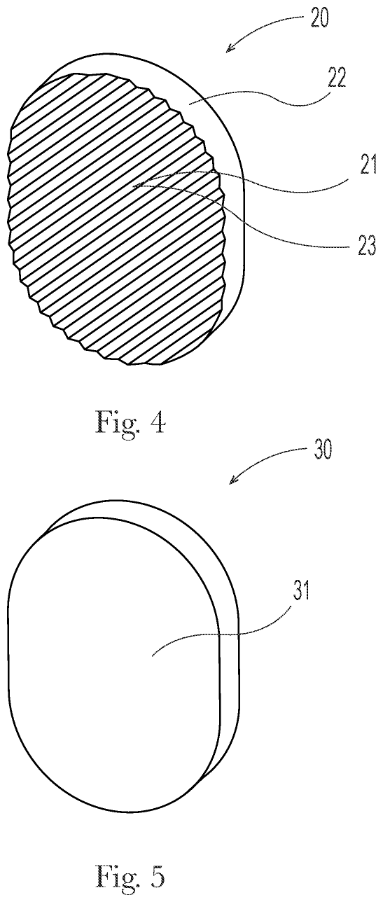

To explain the way a solid article and a visual indicator works to provide the visual product life indicator according to the present invention, it is helpful to understand how the visual indicator is concealed in a first configuration and how shrinkage of the solid article 2 reveals the visual indicator in a second configuration. A method of providing a visual product life signal for a volatile composition dispenser 10 for delivering a volatile composition in an interior space according to the present invention is described with reference to FIGS. 3A, 3B, and 3C.

Specifically, the present invention relates to a method of providing a visual product life signal for a volatile composition dispenser for delivering a volatile composition in an interior space, the method comprising:
providing a volatile composition dispenser comprising a solid article having a volatile composition and a peripheral evaporative surface, wherein the solid article is capable of shrinkage from an initial size to a reduced size smaller than the initial size upon exposure of the solid article to the environment in the interior space; wherein the solid article is characterized by a shrinkage in a direction away from the peripheral evaporative surface of greater than 1% to less than 40%;
providing a visual indicator located proximal to the peripheral evaporative surface of the solid article;
exposing the solid article such that the volatile composition vaporizes for delivering a benefit in the interior space;
wherein the dispenser has a first configuration wherein the visual indicator is not visible, and a second configuration wherein the visual indicator is visible upon shrinkage of the solid article from the initial size to the reduced size for indicating a state of use of the dispenser.

However, the solid article, the visual indicator, and a housing for receiving the solid article may be provided separately in a kit and to be assembled together to form a volatile composition dispenser. Therefore, according to an alternative embodiment, the present invention also relates to a method of providing a visual product life signal for a volatile composition dispenser for delivering a volatile composition in an interior space, the method comprising:
providing a solid article having a volatile composition and a peripheral evaporative surface, wherein the solid article is capable of shrinkage from an initial size to a reduced size smaller than the initial size upon exposure of the solid article to the environment in the interior space; wherein the solid article is characterized by a shrinkage in a direction away from the peripheral evaporative surface of greater than 1% to less than 40%;
providing a visual indicator located proximal to the peripheral evaporative surface of the solid article;
arranging the solid article and the visual indicator in a container to form a volatile composition dispenser for delivering a volatile composition; and
exposing the solid article such that the volatile composition vaporizes for delivering a benefit in the interior space;
wherein the dispenser has a first configuration wherein the visual indicator is not visually perceptible, and a second configuration wherein the visual indicator is visually perceptible upon shrinkage of the solid article from the initial size to the reduced size for indicating a state of use of the dispenser.

Referring to FIG. 3A, the volatile composition dispenser 10 comprises substantially the same components as the volatile composition dispenser 1 of FIG. 1 and differs in that a visual indicator 16 is disposed at a lower part of the housing interior. The volatile composition dispenser 10 comprises a housing 14 for housing a solid article 12, wherein the solid article 12 comprises a volatile composition. The volatile composition dispenser 10 is in a substantially vertical orientation. The solid article 12 has a length L, width W and thickness D defining a three-dimensional self-supporting structure of the solid article 12. A visual indicator 16 is disposed on an interior 15 of the housing 14 behind a central evaporative surface 18 of the solid article 12 and proximal to a peripheral evaporative surface 19 surrounding the central evaporative surface 18. The volatile composition may be described as a perfume comprising a mixture of perfume compounds configured to evaporate from the central evaporative surface 18 and/or the peripheral evaporative surface 19. However, it will be appreciated that any volatile composition capable of vaporizing from a solid phase (i.e., in the solid article 12) to a vapor phase may be employed. The solid article 12 is arranged within the housing 14 such that there is a gap 17 between inner surfaces of the housing and at least a part of the peripheral evaporative surface 19 proximal to the visual indicator 16. The gap 17 may be configured to facilitate air flow in the dispenser so that the peripheral evaporative surface is in fluid communication with air within the housing and the volatile composition can evaporate from the peripheral evaporative surface 19. Specifically, the gap 17 may be configured to enable shrinkage along a first dimension of the solid article at a faster rate relative to shrinkage along a second dimension of the solid article. The first dimension may be one of a length, width, thickness of the solid article, and the second dimension may be the other one of a length, width, thickness of the solid article.

The central evaporative surface 18 is characterized by a central evaporative surface area larger than a peripheral evaporative surface area of the peripheral evaporative surface 19.

FIG. 3A shows the volatile composition dispenser 10 in a first configuration and FIG. 3B is a side section schematic view of FIG. 3A illustrating how the visual indicator 16 may be concealed in a first configuration of the volatile composition dispenser 10. Referring to FIGS. 3A, 3B, the central evaporative surface 18 may comprise at least one region 18A characterized by an optical property selected from the group consisting of: a Haze measurement of at least 50%, a Total Transmission Rate of less than 30%, and combinations thereof. The optical property may be determined at the first configuration or the second configuration according to the measurement method described herein under the Examples. Specifically, in the first configuration before exposure of the solid article 12 to the atmosphere, the visual indicator 16 is concealed by the solid article 12, such as for example, behind the at least a region 18A of the central evaporative surface 18 of the solid article 12. For a solid article that is transparent or semi-transparent or translucent, at least a portion of the solid article may be characterized by a Haze measurement of at least 50%. The surface finish may be a texture such as a matt finish, a 3-dimensional surface texture, or a combination of matt finish and 3-dimensional surface texture. The 3-dimensional surface texture may be defined by a plurality of peaks 181 and valleys 182 on the at least a region 18A of the central evaporative surface 18.

Referring to FIG. 3B, a technical effect of texturing creates an undulating surface on the at least a region 18A of the central evaporative surface 18. Having an undulating surface causes light rays hitting and reflecting different directions to the eye of the user, thereby resulting in light distortion, and rendering the visual indicator not visually perceptible by the human eye.

Referring to FIG. 3A, the solid article 12 has an initial length, L1 and the dispenser 10 is in a first configuration in which the visual indicator 16 is not visible.

Referring to FIG. 3C, upon exposure of the solid article 12 to the environment, the solid article 12 exhibits shrinkage in a direction away from the peripheral evaporative surface 19 and is characterized by a reduced length, L2. The shrinkage is determined based on calculating the difference between L1 and L2 of the solid article 12 wherein L1 is defined by a length between the peripheral evaporative surface 19 to an opposite side 21 to the peripheral evaporative surface 19.

A technical effect of a solid article having a shrinkage of greater than 1% to less than 40% is that a remaining portion of the solid article 12 has sufficient structure for the solid article 12 to be a self-supporting structure which does not require a container for supporting the solid article on a placement surface. Further, it also provides sufficient surface area for ease of removal for users to remove from the placement surface. The placement surface may be a product placement location in the interior space, such as interior surfaces, fixtures, furniture, or the like.

The solid article 12 can be made of any known material that is capable of providing a self-supporting structure and having a mixture of evaporable ingredients and non-evaporable ingredients, as long as the amounts of the evaporable ingredients and non-evaporable ingredients are configured to provide shrinkage of greater than 1% to less than 40%. Evaporable ingredients may include a volatile composition and water. Non-evaporable ingredients may include ingredients suitable for forming a solid article including but not limited to gelling materials, elastomers, polyurethane, or the like.

Table 1 shows an example of a water-based gel composition according to the present invention.

TABLE 1
Example of a Water-based gel composition

| | | Target (%) by |
|---|---|---|
| | | weight of the |
| Ingredient | Function | composition |
| Water | Solvent | 35 |
| Carrageenan and NaCl Mixture | Gelling material | 25 |
| Gellan Gum | Gelling material | 22 |
| Calcium Chloride | Stabilizer | 15 |
| Perfume | Fragrance | 1 |
| Hydrogenated Castor Oil | Emulsifier | 2 |

The solid article may be non-aqueous, preferably comprises less than 1% by weight of water, more preferably substantially free of water.

The solid article may comprise a material selected from the group consisting of: an ethyl cellulose polymer, a chemically cross-linked polyol or derivative thereof, and mixtures thereof.

Table 2 shows an example of a non-aqueous gel composition comprising an ethyl cellulose polymer which is capable of having a shrinkage of 1% to 40%.

TABLE 2
Non-aqueous gel composition

| | Target Range (wt % by |
|---|---|
| Ingredient | weight of the composition) |
| Poly (propylene oxide), weight average | 20 to 75 |
| molecular weight of 300 to 1,000 Daltons | |
| Ethyl cellulose polymer | 0.5 to 30 |
| C₁₋₅ alkyl monoester of a C₈₋₂₂ fatty acid | 20 to 75 |
| Perfume | 4.5 to 50 |
| water | 0 to 1.5 |

The solid article may be a chemically cross-linked polyol, wherein the polyol or derivative thereof is selected from the group consisting of: polyol, polyester polyol, polyglycerol, and mixtures thereof, preferably the polyol derivative is a polyester polyol, more preferably castor oil, even more preferably the polyester polyol is cross-linked using a cross-linking agent selected from the group consisting of: isocyanates, isothiocynates and mixtures thereof.

The solid article may also be molded with a moldable material such as any one of the non-aqueous gel compositions described hereinafter.

### Non-Aqueous Gel Composition:

The gel composition is formed using a cross-linking agent which forms covalent bonds which are stable mechanically and thermally, so once formed are difficult to break. In contrast, physical cross-links rely on changes in the microstructure to achieve stability, such as crystalline regions or regions of high entanglement.

While physical gels can also hold high levels of hydrophobic material such as perfume, the processing of such physical gels is more delicate, as they are more readily broken during manipulation. In addition, such physical gels typically exhibit larger reductions in volume as the hydrophobic material evaporates, in comparison to the cross-linked gels of the present invention, typically at the level of from 50% to 90% by length reduction as the hydrophobic material evaporates. In contrast, the cross-linked gels of the present invention exhibit less shrinkage as the hydrophobic material is released, typically of the order of from 1% to 40%, preferably from 3% to 30%, more preferably from 4% to 20%, or different combinations of the upper and lower percentages described above or combinations of any integer in the ranges listed above, at the end of a time period of 1 to 75 days, preferably from 1 to 60 days, more preferably from 1 to 45 days.

The gel composition can have an elastic modulus G′ of above 0.1 kPa, preferably above 1 kPa, even more preferably above 2 kPa, and below 100 kPa.

The gel can be a chemically cross-linked polyol or derivative thereof. Suitable polyols or derivatives thereof can be selected from the group consisting of: polyol, polyester polyol, polyglycerol, and mixtures thereof. Polyols, polyester polyols, and polyglycerols comprise multiple hydroxyl groups, and are suitable for forming gels having a compact network. In addition, the resultant gel has greater affinity for hydrophobic materials which are less strongly hydrophobic.

Suitable polyols or derivatives thereof can have a molecular weight of from 60 Da to 10000 Da, preferably from 150 Da to 3000 Da, even more preferably from 500 Da to 2000 Da, even more preferably 600 Da to 1300 Da. Longer polyols and derivatives thereof, result in greater flexibility of the gel.

Suitable polyols and derivatives thereof do not comprise terminal hydroxyl groups. Secondary alcohols are particularly suitable. Primary alcohols, having terminal hydroxyl groups, typically result in more linear chains and a more compact network. A combination of primary and secondary alcohols are preferred since they result in a more desired correlation length.

A gel with more optimal pore size is achieved when secondary alcohols are used. Lightly branched polyols and derivatives thereof, such as poly (diethyleneglycol adipates) result in more flexible gels. Preferred polyols and derivatives thereof have at least 2 hydroxyl groups per molecule, more preferably at least 3 hydroxyl groups per molecule.

A polyol is a compound containing multiple hydroxyl groups. Diol polyols, having two hydroxyl-functional groups, result after cross-linking in linear polymers or more open networks having large pore size. In contrast, hydroxyl-functional monomers with functionality larger than two form more compact gels with smaller pore sizes. Suitable polyols include: ethylene glycol, propylene glycol, 1,4-butanediol, 1,6-hexanediol, pentaerythritol, 1,2,6-hexanetriol, 4,6-di-tert-butylbenzene-1,2,3-triol, propanetriol (glycerol), 1,2,5-hexanetriol, 1,2,4-cyclohexanetriol, 2,5-dimethylhexane-1,2,6-triol, 3-hydroxymethylpentane-1,2,5-triol, 1,3,6-hexanetriol, 1,1,5,5-pentanetretraol, 1,2,5,6-hexanetretraol, 1,2,3,4,5,6-hexanehexol (sorbitol) and mixtures thereof. Polyester polyols are hydroxyl-containing esters. Suitable polyester polyols can be selected from the group consisting of: aliphatic polyester polyols, aromatic polyester polyols, organic oil-based polyester polyols, and mixtures thereof. Organic oil-based polyester polyols are preferred. Preferred organic oils are vegetable oils since they typically comprise high levels of unsaturation (C═C bonds) and naturally comprise hydroxyl groups. Suitable polyester polyols include: hexanoic acid, 4-hydroxy-, 1,1′,1″-(1,2,3-propanetriyl) ester; pentanoic acid, 5-amino-4-hydroxy-, 1,1′,1″-(1,2,3-propanetriyl) ester; Polycaprolactone triol; castor oil, hydroxyl sunflower oil (HSO) and mixtures thereof.

Castor oil is particularly suitable. Castor oil (Ricinus oil) is a pale yellow and viscous liquid, derived from the bean of the castor plant (Ricinus communis). Castor oil is predominately made up of triglycerides of fatty acids that contain 87-90% of ricinoleic acid (cis-12-hydroxyoctadec-9-enoic acid) and can be achieved in high purity grades. Castor oil and its derivatives have been used as polyols for polyurethanes and adhesives. The castor oil can be partially hydrogenated. It has been found that castor oil provides the length of the branches and the position of the hydroxyl groups which is particularly suited for providing a chemically cross-linked gel having a pore size which results in slow release of the hydrophobic material, particularly where the hydrophobic material is a perfume. In addition, the chemically cross-linked gels derived from castor oil show less syneresis of the hydrophobic material from the gel.

Polyglycerols are hydroxy-containing ethers. Polyglycerols are typically obtained by the polymerization of alkylene oxides (such as epoxides). Suitable alkylene oxides include ethylene oxide, propylene oxide, butylene oxide, and mixtures thereof, using chain initiators such as ethylene glycol, propylene glycol, diethylene glycol, dipropylene glycol, 1,4-butanediol, neopentyl glycol, pentaerythritol, hexanetriol, sorbitol, glycerol, and mixtures thereof. Suitable polyglycerols can be selected from the group consisting of: α, α-diglycerol, α, β-diglycerol, hyperbranched polyglycerol, dendritic polyglycerol, and mixtures thereof. Hyperbranched polyglycerols are aliphatic polyethers with multiple hydroxyl end groups that are obtained from the nonsymmetric polyaddition of glycidol to glycerol resulting in a globular branch-on-branch structure which provides special internal flexibility. Dendritic polyglycerols are a hyperbranched polyglycerol with a well-defined symmetric and spherical three-dimensional structure around a core. Apart from improving gel elasticity, the dendritic structure with sterically shielded core together with the exceptionally high number of functional groups of hyperbranched polyglycerols produces flexible gels with relatively low pore size, which increase the longevity of final composition by reducing the diffusion rate not only as a consequence of physically entrapping the hydrophobic material, but also enhancing H-bonding and Van der Waals interactions. Such polyglycerols can be purchased from Nanopartica GmbH (Germany) and Sigma-Aldrich. Suitable polyglycerols include: polyethylene glycol, polypropylene glycol, poly (diethylene glycol), poly (dipropylene glycol), poly(1,4-butanediol), poly (neopentyl glycol), poly(1,6-hexanediol), and mixtures thereof. The polyglycerol preferably has from 2 to 50, preferably from 4 to 30 repeat units.

Any suitable cross-linking agent can be used, though cross-linking agents selected from the group consisting of: isocyanates, isothiocynates and mixtures thereof, are preferred. The cross-linking agent can be a linear, branched, or cyclic isocyanate, and mixtures thereof. Cyclic isocyanates and mixtures thereof are preferred. Suitable cyclic isocyanates include heterocyclic isocyanates such as 1,3,5-tris(5-isocyanatopentyl)-1,3,5-triazine-2,4,6(1H,3H,5H)-trione.

Suitable cross-linking agents can be selected from the group consisting of: 1, 4-butane diisocyanate (BDI), 1,6 hexamethylene diisocyanate (HMDI), L-Lysine ethyl ester diisocyanate (LDI), 4,4′-Methylenebis(cyclohexyl isocyanate) (H12MDI), Glycolide-ethylene glycol-Glycolide isocyanate (Bezwada, LLC), 4,4′-Methylenebis(phenyl isocyanate) (MDI), 2,4′-Methylenebis(phenyl isocyanate) (MDI), 2,2′-Methylenebis(phenyl isocyanate) (MDI), Isophorone diisocyanate (IPDI), 2,4-toluene diisocyanate (2,4-TDI), 2,6-toluene diisocyanate (2,6-TDI), Poly (hexamethylene diisocyanate) (PDI), 1,3-bis(2-isocyanatopropan-2-yl)benzene, Poly (pentamethylene diisocyanate) and mixtures thereof, preferably 1,6 hexamethylene diisocyanate (HMDI), L-Lysine ethyl ester diisocyanate (LDI), Poly (pentamethylene diisocyanate), Poly (hexamethylene diisocyanate) (PDI), 1,3,5-tris(5-isocyanatopentyl)-1,3,5-triazine-2,4,6(1H,3H, 5H)-trione, and mixtures thereof. Such cross-linking agents are available from Sigma-Aldrich and from Covestro under trade name of Desmodur® eco N 7300.

The cross-linking agent can have a viscosity below 2.500 mPa·s at 25° C. and an isocyanate equivalent weight of from 15% to 40%, preferably from 18% to 30%. Such cross-linking agents are more easily blended with the polyol. As a result, more uniform gels can be achieved.

The gel is preferably essentially free, or free of unreacted isocyanates and/or isothiocyanates.

The gel can further comprise a hydroxyl containing polymer, a hydroxyl containing oligomer or mixtures thereof. The hydroxyl containing polymer and/or oligomer can be used to alter the elasticity of the gel composition, and therefore the longevity of the perfume release. since a higher elastic modulus G′ slows the perfume release.

Suitable hydroxyl containing polymers can be selected from the group consisting of: poloxamers, gelatins, carrageenan, chitin, chitosan, and mixtures thereof.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Suitable poloxamers have a weight average molecular weight of from 1500 g/mol to 15000 g/mol and a poly(ethylene oxide) weight percentage of from 10% to 80%, preferably from 50% to 80%. Suitable poloxamers are commercially available under the tradename of Pluronic® from BASF.

Gelatins are typically translucent, colorless, and typically obtained from collagen from various animal body parts. They are commonly used as a gelling agent in food, pharmaceutical industry, vitamin capsules, photography, and cosmetic manufacturing. Suitable gelatines can have a bloom of from 90 to 300. Bloom is a test to measure the strength of a gel or gelatin and is measured according to the method outlined by Bloom in U.S. Pat. No. 1,540,979. The test determines the weight in grams needed by a plunger with a diameter of 0.5 inch (12.7 mm) to depress the surface of the gel 4 mm without breaking it, at a temperature of 25° C. The result is expressed in Bloom (grades). It is usually between 30 and 300 Bloom. To perform the Bloom test on gelatin, a 6.67% by weight gelatin solution is kept for 17-18 hours at 10° C. prior to being tested.

Carrageenan are sulfated polysaccharide for instance derived from red algae, commonly known as Irish moss. They are typically composed principally of alpha-D-galactopyranose-4-sulfate units and 3,6-anhydro-alpha-D-galactopyranose units. At least three forms are known, designated, respectively, as “iota”, “kappa” and “lambda” carrageenan which differ in the ratios of the two galactopyranose units and accordingly in their sulfate ester content.

Kappa-carrageenan is the principal component in aqueous extracts from Chondrus crispus and Gigartina stellata. It is lower in sulfate ester content than iota and lambda carrageenan.

Chitosan is typically obtained by deacetylation under alkaline conditions of chitin, which is the second most abundant biopolymer in nature, after cellulose. Chitin can be found as an important constituent of the exoskeleton in animals, especially in crustaceans, molluscs, and insects, and it is also the principal polymer in the cell wall of certain fungi. Chitin and chitosan are linear polysaccharides composed of randomly distributed β-(1-4)-linked D-glucosamine (deacetylated unit) and N-acetyl-D-glucosamine (acetylated unit). Chitosan has two types of reactive groups that can be grafted: the free amine groups on the deacetylated units and the hydroxyl groups on the C3 and C6 carbons on acetylated or deacetylated units.

The chitosan of the present invention may have a molecular weight from 10,000 g/mol to 4,000,000 g/mol, preferably from 70,000 g/mol to 1,600,000 g/mol. Suitable chitosan may have a degree of de-acetylation of at least 50%, preferably at least 60%, more preferably at least 70%, even more preferably at least 75%.

The gel composition can be transparent or even translucent. The gel composition can have any suitable shape, such as star, circular or pyramidal. The gel composition may be colored by adding dye. The gel compositions of the present invention can be molded or even 3D printed to the desired shape.

The gel composition can be any suitable shape or size since both define the evaporative surface area of the gel composition. It is known that the shape and size of the gel composition can affect the release and longevity of the hydrophobic material. For instance, thin sheets result in faster release and lower longevity than spheres of the same mass of the gel composition. Suitable gel compositions can have an evaporative surface area of less than 150 cm², preferably from 3.0 to 100 cm², more preferably from 6.0 to 60 cm².

The evaporative surface area can be measured by creating a 3D model of the gel composition using CAD software and using the CAD software to calculate the surface area. Any suitable CAD software can be used, such as AutoCAD® 2013.

FIG. 4 is a front perspective view of a solid article 20 for a volatile composition dispenser according to the present invention.

Specifically, the solid article 20 comprises the same features as the solid article 1 of FIG. 1 and comprises a central evaporative surface 21 and a peripheral evaporative surface 22 surrounding the central evaporative surface 21. The central evaporative surface 21 comprises a 3-dimensional pattern 23 defining an undulating surface covering all of the central evaporative surface 21. The solid article 20 may be characterized by a Haze measurement of at least 50% for concealing a visual indicator in a first configuration of the volatile composition dispenser as described hereinbefore under the Method.

FIG. 5 is a front perspective view of a different design of a solid article 30 having a similar size and shape to the solid article 20 of FIG. 3 but without a textured surface. The solid article 30 has substantially the same features as the solid article 20 of FIG. 3 except that the solid article 30 is substantially free of a textured surface. To conceal a visual indicator in the first configuration, the solid article 30 may characterized by a Total Transmission Rate of less than 30%. The Total Transmission Rate of less than 30% of the solid article 30 may be obtained by adding colored dye during the making of the solid article 30. Techniques of adding colored dye depends on a structural material used for making the solid article 30. The structuring material include components for making a gel composition described hereinbefore.

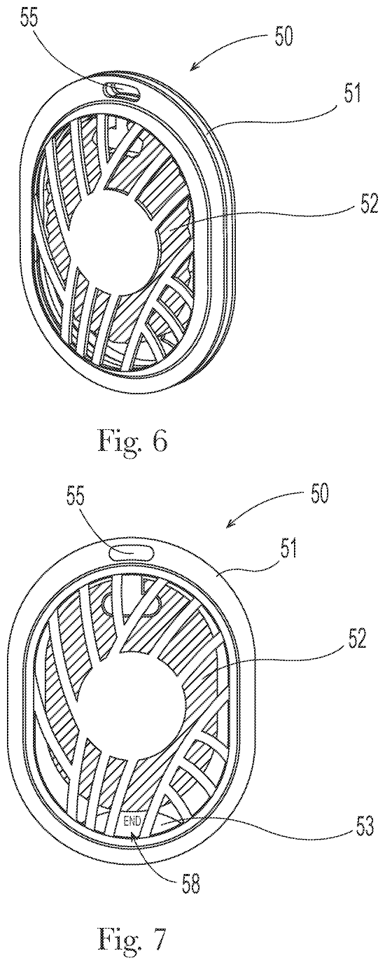

FIG. 6 is a front perspective view of an alternative embodiment of a volatile composition dispenser 50 comprising a housing 51 with a solid article 52 according to the present invention. In FIG. 6, the solid article 52 has an initial size and a visual indicator 58 in a first configuration in which the visual indicator 58 is not visually perceptible according to the present invention.

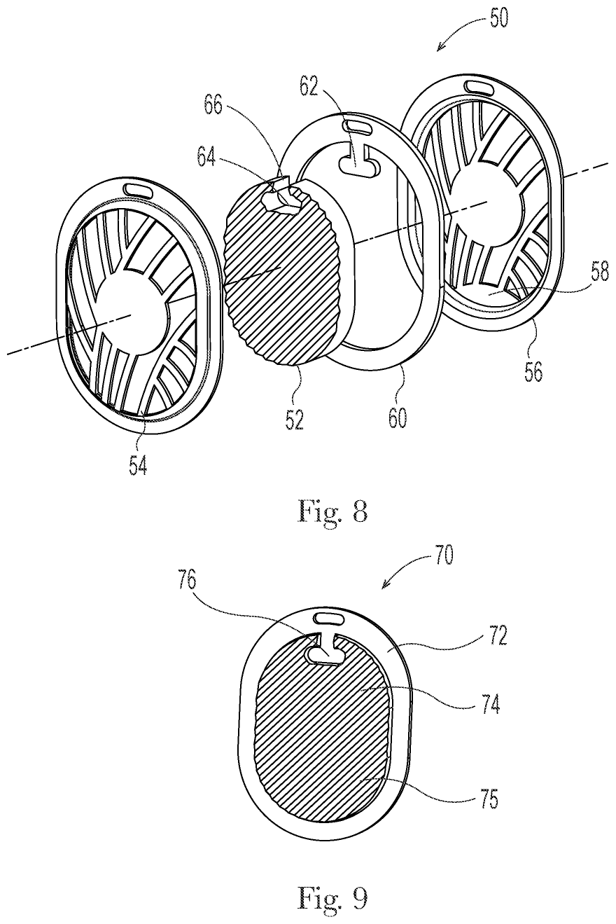

FIG. 7 is a front view of the volatile composition dispenser 50 of FIG. 6 in a second configuration in which the visual indicator 58 is visually perceptible. Specifically, the housing 51 further comprises an inner surface 53, wherein the visual indicator 58 is disposed on the inner surface 53.

FIG. 8 is a perspective view of components in the volatile composition dispenser 50 of FIG. 6. The housing 51 may comprise a front cover 54 and a rear frame 56, which upon assembly, the front cover 54 and the rear frame 56 define a housing interior for receiving the solid article 52. An article holder 60 may be disposed between the front cover 54 and rear cover 56 wherein the article holder 60 is configured for holding the solid article 52 in a predetermined position within the housing. Each of the front cover 54 and the rear frame 56 is provided with a plurality of openings 403 (hereinafter “opening”) for vaporizing of volatile composition in the solid article 52 into the interior space. The article holder 60 comprises an anchor 62 for releasably attaching the solid article 52 to the article holder 60, and a center opening 64 configured for circumscribing the solid article 52. The solid article 52 may comprise an aperture 64 sized and shaped to mate with the anchor 62. Specifically, the aperture 64 extends through an end part 66 of the solid article 52.

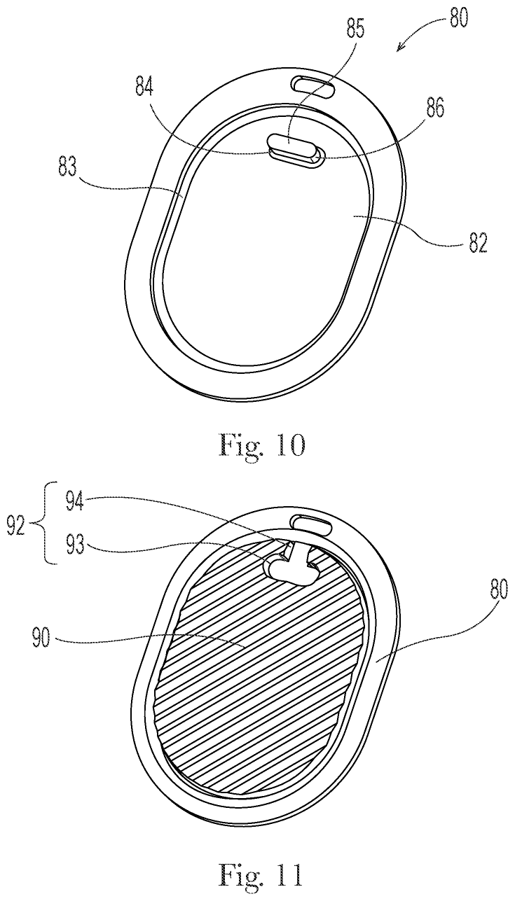

FIG. 9 is a front perspective view of an alternative embodiment of a volatile composition dispenser 70 with an article holder 72 and a solid article 74 according to the present invention. Specifically, the dispenser 70 has substantially the same features as the dispenser 50 of FIG. 8 and differs from the dispenser 50 in that there is no housing. The article holder 72 comprises an anchor 76 for supporting the solid article 74 in a hanging configuration above a surface in an interior space, wherein the solid article 74 is releasably attached to the anchor 76. A central evaporative surface 75 of the solid article 74 is front facing and a visual indicator (not shown) may be provided on an opposite side to the central evaporative surface 75.

FIG. 10 is a front perspective view of an alternative embodiment of an article holder 80 for a volatile composition dispenser according to the present invention. The article holder 80 comprises a bottom surface 82 and a side wall 83 extending from the bottom surface 82 to define an internal cavity for receiving a solid article. An anchor 84 is disposed on the bottom surface 83 for releasably attaching the solid article to the article holder 80. The anchor 84 may comprise a protrusion extending from the bottom surface 83. The anchor 84 comprises an anchor top 85 and an anchor side 86 circumscribing the anchor top 85.

FIG. 11 is a front perspective view of the article holder 80 of FIG. 10 with a solid article 90 according to the present invention. The solid article 90 comprises an anchor receiving aperture 92 for releasable attachment to the anchor 84. The anchor receiving aperture 92 may comprise a first aperture portion 93 having a size and shape corresponding to a size and shape of the anchor 84. The anchor receiving aperture 92 may further comprise a second aperture portion 94 extending from the first aperture portion 93 through an outer periphery of the solid article 90.

The housing, front cover, rear cover, and the article holder may be made of plastic, paper, or any material chemically compatible with the solid article.

The following examples are intended to more fully illustrate the present invention and are not to be construed as limitations of the present invention since many variations thereof are possible without departing from the scope of the present invention. All parts, percentages and ratios used herein are expressed as percent weight unless otherwise specified.

### Examples

Data is provided demonstrating the volatile composition dispenser of the present invention having improved visible indicator to inform consumers of the product life signal. Solid articles according to the present invention are prepared based on the composition details described in Table 1 below and are evaluated for the respective shrinkage (results shown in Table 2).

TABLE 1

| | | Inventive Sample (% by weight | | | | | |
|---|---|---|---|---|---|---|---|
| | | of the solid article) | | | | | |
| Component | Material | 1 | 2 | 3 | 4 | 5 | 6 |
| Polyester | Castor oil | 29 | 29 | 29 | 29 | 29 | 29 |
| Polyol | | | | | | | |
| Cross-linking | Isocyanate | 21 | 21 | 21 | 21 | 21 | 21 |
| Agent | | | | | | | |
| Volatile | Mixture of | 50 | 50 | 50 | 50 | 50 | 30 |
| Composition | perfume | | | | | | |
| | raw | | | | | | |
| | materials | | | | | | |
| Total | | 100 | 100 | 100 | 100 | 100 | 100 |

Inventive Samples of solid articles made with the composition details described in Table 1 according to the process described below.

### Process of Making:

The polyol or derivative thereof, is mixed with the volatile composition, and optionally a hydroxyl containing polymer is added. Then, the cross-linking agent is added at a temperature from 5° C. to 35° C., preferably 15° C. to 30° C. and further mixed in order to provide a homogeneous mixture. The mixture is poured into a mould of the desired shape, and cured, preferably at a curing temperature from 20° C. to 30° C. Such temperatures limit the evaporation of volatile components of the hydrophobic material. Alternatively, the mixture can be kept at 5° C. or less, in order to avoid curing. Curing will then start only once the temperature is raised to the curing temperature.

The polyol or derivative thereof can be mixed with the cross-linking agent, and optionally a hydroxyl containing polymer, preferably at a temperature from 20° C. to 85° C., more preferably from 30° C. to 75° C., for 10 min to 10 hours, preferably from 15 min to 2 hours. The mixture is cooled down and the hydrophobic material is added preferably at a temperature from 10° C. to 40° C., more preferably 15° C. to 30° C. and can be further mixed, for instance, from 15 to 120 min. The mixture is poured into the desired mould and cured, preferably at a temperature from 20° C. to 30° C.

Alternatively, all of the components of the gel composition can be blended at a low temperature, such as 5° C. or less, before the temperature is increased to the curing temperature.

### Results

Shrinkage results of the Inventive Samples 1 to 6 of Table 1 are shown in Table 2 below. Specifically, the shrinkage is determined based on Formula (1) below:

Shrinkage=(Initial Length−Reduced Length)/Initial Length×100%

wherein,
Initial Length=length or diameter of the solid article before use
Reduced Length=length or diameter of the solid article after a predetermined time period of use.

TABLE 2

| | | | | Shrinkage (of | | |
|---|---|---|---|---|---|---|
| | | | | Length/Diameter) At | | |
| Inventive | Sample | Initial Dimensions (first | Weight | 45 Days after use | | % Shrinkage |
| Sample | Shape | configuration before use) | (g) | (second configuration) | | at 45 days |
| 1 | Circle | Diameter = 40 mm | 5.8 | 4.2 | mm | 10.5 |
| | | Thickness = 5 mm | | | | |
| 2 | Oval | Length × Width × | 5.8 | 5.4 | mm | 10.0 |
| | | Thickness = | | | | |
| | | 54 × 26.5 × 5 mm | | | | |
| 3 | Oval | Length × Width × | 8.2 | 5.6 | mm | 10.4 |
| | | Thickness = | | | | |
| | | 54 × 26.5 × 5 mm | | | | |
| 4 | Rectangle | Length × Width = | 8.2 | 7 | mm | 12.5 |
| | | 56 × 32 × 5 mm | | | | |
| 5 | Rectangle | Length × Width = | 8.2 | 5 | mm | 10.2 |
| | | 49 × 27 × 5 mm | | | | |
| 6 | Rectangle | Length × Width = | 8.2 | 3.1 | mm | 6.3 |
| | | 49 × 27 × 5 mm | | | | |

All the above Inventive Samples 1 to 6 have a shrinkage of 6.3% to 12.5%, i.e., less than 40%. As a result, each of the samples at the end of life is a single unitary piece having a size sufficient for easy removal and thereby reducing mess. In contrast, conventional gels have a gelling polymer which remains after water and perfume evaporates, and the remaining gelling polymer causes stickiness in the gel. Further, the remaining gel also stick to the container break easily and causes messy residue.

Solid articles according to the present invention are prepared based on the composition details described in Table 3 below and according to the process described hereinbefore. The solid articles are evaluated for the visual perceptibility of the visual indicator in the first configuration (results shown in Table 4).

TABLE 3

| | | Comparative | | |
|---|---|---|---|---|
| | | Sample | Inventive Sample | |
| | | (% by weight of | (% by weight of | |
| | | the solid article) | the solid article) | |
| | Material | #7 | #8 | #9 |
| Component | | | | |
| Polyester | Castor oil | 29 | 29 | 29 |
| Polyol | | | | |
| Cross-linking | Isocyanate | 21 | 21 | 21 |
| Agent | | | | |
| Volatile | Mixture of | 50 | 50 | 50 |
| Composition | perfume raw | | | |
| | materials | | | |
| Additional | | | | |
| Parameters | | | | |
| Surface Finish | | No texture | Textured | No texture |
| Color Dye | | 0 | 0 | 3% |

TABLE 4

| | | | | Is the visual |
|---|---|---|---|---|
| | | Diffuse | | indicator visually |
| | Total | Trans- | | perceptible or |
| | Transmis- | mis- | | noticed in the first |
| | sion (%) | sion | Haze (%) | configuration prior |
| Example | (< or = 30%) | (%) | (> or = 50%) | to shrinkage? |
| Comparative | 91.7 | 4.2 | 4.5 | Yes |
| Sample 7 | | | | |
| Inventive | 76.9 | 72.9 | 94.8 | No |
| Sample 8 | | | | |
| Inventive | 21.5 | 0.7 | 3.5 | No |
| Sample 9 | | | | |

The total transmission, diffuse transmission, and haze measurements for each of the Comparative and Inventive Samples are measured according to the ASTM 01003-13 test method for Haze with PerkinElmer Lambda 950 UV/VIS/NIR spectrophotometer (with 150 mm integrating sphere) instrument. A low Haze measurement value corresponds to a clarity of the solid article.

Specifically, Haze is determined by the following equation:

Haze=Diffuse Transmission/(Total Transmission(specular and diffuse transmission))*100%

Referring to Table 4, Comparative Sample 7 has a Total Transmission much greater than 30% and a Haze measurement much smaller than 50%. As such, the visual indicator is still visually perceptible relative to Inventive Samples 8 and 9.

In an example, there is:
A. A volatile composition dispenser comprising:

a solid article comprising a volatile composition and a peripheral evaporative surface, wherein the solid article is capable of shrinkage relative to the peripheral evaporative surface from an initial size to a reduced size smaller than the initial size upon exposure of the solid article to the environment in an interior space wherein the solid article is characterized by a shrinkage in a direction away from the peripheral evaporative surface of greater than 1% to less than 40%; and a visual indicator located proximal to the peripheral evaporative surface of the solid article;

wherein the dispenser has a first configuration wherein the visual indicator is not visually perceptible, and a second configuration wherein the visual indicator is visually perceptible upon shrinkage of at least a portion of the solid article from the initial size to the reduced size for indicating a state of use of the dispenser.
B. The dispenser according to paragraph A, wherein the shrinkage is from 3% to 30%, preferably from 4% to 20%, at the end of a time period of 1 to 75 days, preferably from 1 to 60 days, more preferably from 1 to 45 days.
C. The dispenser according to paragraphs A or B, further comprising a housing configured for receiving the solid article, and a gap between the peripheral evaporative surface of the solid article and an inner surface of the housing, wherein the gap is configured to enable shrinkage along a first dimension of the solid article at a faster rate relative to shrinkage along a second dimension of the solid article.
D. The dispenser according to paragraph C, further comprising an anchor arranged within the housing, wherein the solid article is releasably attached to the anchor to provide the gap.
E. The dispenser according to any one of paragraphs A to D, wherein the solid article comprises a central evaporative surface and the visual indicator is disposed behind the central evaporative surface.
F. The dispenser according to any one of paragraphs A to E, wherein the visual indicator indicates a state of the dispenser selected from the group consisting of: an end of life of the dispenser, a quantitative state indicative of a number of days use of the dispenser, and combinations thereof, preferably the visual indicator is a set of characters or graphical symbols indicative of the state of the dispenser, more preferably, the visual indicator is a single word or a graphical symbol indicative of an end of life of the dispenser.
G. The dispenser according to any one of paragraphs A to F, wherein the volatile composition is selected from the group consisting of: a perfume, a deodorizing agent, a sanitizing agent, an insect repellant, a malodor reduction agent, and mixtures thereof, preferably the volatile composition is present in a level of 3 wt % to 85 wt %, preferably from 15 wt % to 75 wt %, more preferably from 20 wt % to 60 wt % of the solid article.
H. The dispenser according to any one of paragraphs A to G, wherein the solid article is non-aqueous, preferably comprises less than 1% by weight of water, more preferably substantially free of water.
I. The dispenser according to any one of paragraphs A to H, wherein the solid article comprises a gel composition selected from the group consisting of: an ethyl cellulose polymer, a chemically cross-linked polyol or derivative thereof, and mixtures thereof.
J. The dispenser according to paragraph I wherein the solid article comprises a chemically cross-linked polyol, wherein the polyol or derivative thereof is selected from the group consisting of: polyol, polyester polyol, polyglycerol and mixtures thereof, preferably the polyol derivative is a polyester polyol, more preferably castor oil, even more preferably the polyester polyol is cross-linked using a cross-linking agent selected from the group consisting of: isocyanates, isothiocynates and mixtures thereof.
K. The dispenser according to any one of paragraphs A to J, wherein at least a portion of the solid article is characterized by an optical property selected from the group consisting of: a Haze measurement of at least 40%, a Total Transmission Rate of less than 30%, and combinations thereof.
L. The dispenser according to any one of paragraphs D to K, wherein the anchor is comprised in an article holder.
M. The dispenser according to paragraph L, wherein the article holder comprises a center opening configured for receiving the solid article.
N. A solid article for a volatile composition dispenser, the solid article comprising:

a volatile composition, at least one central evaporative surface, and a peripheral evaporative surface circumscribing the central evaporative surface, wherein the solid article is capable of shrinkage relative to the peripheral evaporative surface from an initial size to a reduced size smaller than the initial size upon exposure of the solid article to the environment in an interior space wherein the solid article is characterized by a shrinkage in a direction away from the peripheral evaporative surface of greater than 1% to less than 40%; wherein the central evaporative surface is characterized by a central evaporative surface area of less than 150 cm², preferably from 3.0 to 100 cm², more preferably from 6.0 to 60 cm².
O. A method of providing a visual product life signal for a volatile composition dispenser for delivering a volatile composition in an interior space, the method comprising:

providing a volatile composition dispenser comprising a solid article having a volatile composition and a peripheral evaporative surface, wherein the solid article is capable of shrinkage from an initial size to a reduced size smaller than the initial size upon exposure of the solid article to the environment in the interior space; wherein the solid article is characterized by a shrinkage in a direction away from the peripheral evaporative surface of greater than 1% to less than 40%;

providing a visual indicator located proximal to the peripheral evaporative surface of the solid article; and
exposing the solid article such that the volatile composition vaporizes for delivering a benefit in the interior space;

wherein the dispenser has a first configuration wherein the visual indicator is not visually perceptible, and a second configuration wherein the visual indicator is visually perceptible upon shrinkage of the solid article from the initial size to the reduced size for indicating a state of use of the dispenser.
P. A method of providing a visual product life signal for a volatile composition dispenser for delivering a volatile composition in an interior space, the method comprising:
   providing a solid article having a volatile composition and a peripheral evaporative surface, wherein the solid article is capable of shrinkage from an initial size to a reduced size smaller than the initial size upon exposure of the solid article to the environment in the interior space; wherein the solid article is characterized by a shrinkage in a direction away from the peripheral evaporative surface of greater than 1% to less than 40%;
   providing a visual indicator located proximal to the peripheral evaporative surface of the solid article;
   arranging the solid article and the visual indicator in a container to form a volatile composition dispenser for delivering a volatile composition; and
   exposing the solid article such that the volatile composition vaporizes for delivering a benefit in the interior space;
   wherein the dispenser has a first configuration wherein the visual indicator is not visually perceptible, and a second configuration wherein the visual indicator is visually perceptible upon shrinkage of the solid article from the initial size to the reduced size for indicating a state of use of the dispenser.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A volatile composition dispenser comprising:
a solid article comprising a volatile composition and a peripheral evaporative surface, wherein the solid article is capable of shrinkage relative to the peripheral evaporative surface from an initial size to a reduced size smaller than the initial size upon exposure of the solid article to the environment in an interior space wherein the solid article is characterized by a shrinkage in a direction away from the peripheral evaporative surface of greater than 1% to less than 40%; and
a visual indicator located proximal to the peripheral evaporative surface of the solid article;
wherein the dispenser has a first configuration wherein the visual indicator is not visually perceptible, and a second configuration wherein the visual indicator is visually perceptible upon shrinkage of at least a portion of the solid article from the initial size to the reduced size for indicating a state of use of the dispenser.

2. The dispenser according to claim 1, wherein the shrinkage is from 3% to 30%, at the end of a time period of from 1 to 75 days.

3. The dispenser according to claim 1, further comprising a housing configured for receiving the solid article, and a gap between the peripheral evaporative surface of the solid article and an inner surface of the housing, wherein the gap is configured to enable shrinkage along a first dimension of the solid article at a faster rate relative to shrinkage along a second dimension of the solid article.

4. The dispenser according to claim 3, further comprising an anchor arranged within the housing, wherein the solid article is releasably attached to the anchor to provide the gap.

5. The dispenser according to claim 1, wherein the solid article comprises a central evaporative surface and the visual indicator is disposed behind the central evaporative surface.

6. The dispenser according to claim 1, wherein the visual indicator indicates a state of the dispenser selected from the group consisting of: an end of life of the dispenser, a quantitative state indicative of a number of days use of the dispenser, and combinations thereof.

7. The dispenser according to claim 1, wherein the volatile composition is selected from the group consisting of: a perfume, a deodorizing agent, a sanitizing agent, an insect repellant, a malodor reduction agent, and mixtures thereof, and wherein the volatile composition is present in a level of 3 wt % to 85 wt %, of the solid article.

8. The dispenser according to claim 1, wherein the solid article is non-aqueous, and comprises less than 1% by weight of water.

9. The dispenser according to claim 1, wherein the solid article comprises a gel composition selected from the group consisting of: an ethyl cellulose polymer, a chemically cross-linked polyol or derivative thereof, and mixtures thereof.

10. The dispenser according to claim 9, wherein the solid article comprises a chemically cross-linked polyol, wherein the polyol or derivative thereof is selected from the group consisting of: polyol, polyester polyol, polyglycerol and mixtures thereof.

11. The dispenser according to claim 1, wherein at least a portion of the solid article is characterized by an optical property selected from the group consisting of: a Haze measurement of at least 40%, a Total Transmission Rate of less than 30%, and combinations thereof.

12. The dispenser according to claim 4, wherein the anchor is comprised in an article holder.

13. The dispenser according to claim 12, wherein the article holder comprises a center opening configured for receiving the solid article.

14. The dispenser according to claim 1, wherein the shrinkage is from 4% to 20%, at the end of a time period of from 1 to 75 days.

15. The dispenser according to claim 6, wherein the visual indicator comprises a set of characters or graphical symbols indicative of the state of the dispenser.

16. The dispenser according to claim 6, wherein the visual indicator comprises a single word or a graphical symbol indicative of an end of life of the dispenser.

17. The dispenser according to claim 1, wherein the volatile composition is selected from the group consisting of: a perfume, a deodorizing agent, a sanitizing agent, an insect repellant, a malodor reduction agent, and mixtures thereof, and wherein the volatile composition is present in a level of from 15 wt % to 75 wt % of the solid article.

18. The dispenser according to claim 10, wherein the solid article comprises a polyol derivative comprising a polyester polyol.

19. A solid article for a volatile composition dispenser, the solid article comprising:
a volatile composition, at least one central evaporative surface, and a peripheral evaporative surface circumscribing the central evaporative surface, wherein the solid article is capable of shrinkage relative to the peripheral evaporative surface from an initial size to a reduced size smaller than the initial size upon exposure of the solid article to the environment in an interior space wherein the solid article is characterized by a shrinkage in a direction away from the peripheral evaporative surface of greater than 1% to less than 40%; wherein the central evaporative surface is characterized by a central evaporative surface area of less than 150 cm².

20. A method of providing a visual product life signal for a volatile composition dispenser for delivering a volatile composition in an interior space, the method comprising:
providing a solid article having a volatile composition and a peripheral evaporative surface, wherein the solid article is capable of shrinkage from an initial size to a reduced size smaller than the initial size upon exposure of the solid article to the environment in the interior space; wherein the solid article is characterized by a shrinkage in a direction away from the peripheral evaporative surface of greater than 1% to less than 40%;
providing a visual indicator located proximal to the peripheral evaporative surface of the solid article;
arranging the solid article and the visual indicator in a container to form a volatile composition dispenser for delivering a volatile composition; and
exposing the solid article such that the volatile composition vaporizes for delivering a benefit in the interior space;
wherein the dispenser has a first configuration wherein the visual indicator is not visually perceptible, and a second configuration wherein the visual indicator is visually perceptible upon shrinkage of the solid article from the initial size to the reduced size for indicating a state of use of the dispenser.
